(19) United States
(12) Patent Application Publication
(10) Pub. No.: US 2025/0213187 A1
(43) Pub. Date: Jul. 3, 2025

(54) METHOD FOR EVALUATING BODY COMPOSITION AND SYSTEM FOR USING THEREOF

(71) Applicants: National Cheng Kung University, Tainan (TW); National Cheng Kung University Hospital, Tainan (TW)

(72) Inventors: Pei-Fang SU, Tainan (TW); Yi-Shan TSAI, Tainan (TW); Yu-Hsuan LAI, Tainan (TW); Yi-Chia LIU, Tainan (TW)

(21) Appl. No.: 18/942,902

(22) Filed: Nov. 11, 2024

Related U.S. Application Data
(60) Provisional application No. 63/548,395, filed on Nov. 14, 2023.

(51) Int. Cl. A61B 5/00

(52) U.S. Cl. CPC A61B 5/4869; A61B 5/0077; A61B 5/7275

(57) ABSTRACT

The present invention provides a method for evaluating body composition and a system for using thereof. The method comprises inputting a body composition index and an individual variable to a body composition prediction model so as to produce a body composition evaluation index. The body composition index, comprising a muscle index, a fat index or a comprehensive index, is measured according to a medical image, and the individual variable corresponds to an individual variable of the medical image. The system is configured to obtain the body composition index and the individual variable, and produces the body composition evaluation index accordingly. By using the method and the system, a quantile and a body age evaluation value of a corresponding individual among a normal population can be obtained, and requires only a medical image or a body composition index produced according thereto, which assists judgement of the individual's health status.

### CROSS-REFERENCE TO RELATED APPLICATION

This application claims the benefit of provisional application Ser. No. 63/548,395, filed Nov. 14, 2023. The disclosure of the above application is incorporated herein in its entirety by reference.

### FIELD OF THE INVENTION

The present invention is related to body composition analysis, specifically related to mass or density analysis of muscle, bone, or fat, and, in particular, being related to measurement of body composition through imaging analysis; a normal model is established based on measurement of body compositions in the present invention, and thereby predicting a quantile range of subject's body composition in a normal distributed group; additionally, body age of a subject is also estimated according to the normal model of body composition, combined with results of mass or density analysis of muscle, bone or fat.

### BACKGROUND OF THE INVENTION

In body composition, the ratio of skeletal muscle to fat is one of the key indicators for assessing individual health. Among these, skeletal muscle, as an endocrine and paracrine organ, plays a crucial role in the interaction between skeletal muscle mass and other organs such as the liver, adipose tissue, pancreas, bones, and the cardiovascular system through the secretion of muscle-derived proteins (e.g., myokines).

With increasing age, the inevitable reduction in physical activity leads to a decrease in muscle mass, which reduces metabolic function and increases the risk of metabolic diseases such as obesity, insulin resistance, hyperglycemia, dyslipidemia, hypertension, or non-alcoholic fatty liver disease.

Moreover, muscle health is linked to immune function. Studies have shown that limb muscle mass is negatively correlated with blood levels of C-reactive protein (CRP) and IL-6, indicating that reduced muscle mass indirectly contributes to chronic inflammation. On the other hand, research has also shown that the expression of genes regulating skeletal muscle mitochondrial function, oxidative capacity, and glucose metabolism significantly declines with age, accelerating individual aging. In other words, muscle mass can be considered an individual's health reserve.

Existing techniques include methods to directly or indirectly assess muscle mass, such as imaging analysis through ultrasound or computed tomography (CT) scans, indirect measurement using the deuterated creatine dilution method, or estimating muscle mass based on calf circumference. These methods are used to determine whether to introduce nutritional interventions, muscle function training, or other therapeutic measures to improve individual health.

### SUMMARY OF THE INVENTION

Nonetheless, the existing techniques only provide muscle mass or fat mass of a single individual, and clinical significance of such mass indices still requires evaluation by a physician. Precise determination of correlation between body composition and health status of an individual can not be implemented with insufficient data volume. In light of this, a body composition normal model based on large volume of body composition data is in urgent need, by which health evaluation result can be produced rapidly and precisely, based on body composition data and individual information corresponding thereto.

In one aspect, the present invention provides a method for evaluating body composition, comprising: inputting a body composition index and an individual variable to a body composition prediction model so as to produce a body composition evaluation index, wherein the body composition evaluation index comprises a body composition quantile value or a body age evaluated value, and wherein the body composition index is measured based on a medical image, and comprises a muscle index, a fat index or a comprehensive index, and the individual variable corresponds to an individual variable of the medical image; and outputting the body composition evaluation index.

Preferably, the body composition prediction model is defaulted to contain m prediction quantile values and a prediction index matrix, wherein the prediction index matrix is a matrix of dimension m*1 comprising m prediction body composition index corresponding to the prediction quantile values one on one, and m is any one of positive integers larger or equal to 1, wherein the method further comprises: comparing the body composition index and the prediction body composition index based on the prediction index matrix so as to obtain a corresponding predicted quantile value outputted to be the body composition quantile value.

Preferably, the prediction index matrix is derived from a multiplication of a prediction coefficient matrix and an individual variable matrix, and the prediction coefficient matrix is a matrix of dimension m*(n+1) comprising a prediction constant corresponding to each one of the prediction quantile values and a prediction coefficient set corresponding to each one of the prediction quantile values, and wherein the prediction coefficient set comprises n prediction coefficients, and n is any one of positive integers larger or equal to 1; the individual variable matrix is a matrix of dimension (n+1)*1 comprising a prediction variable corresponding to the prediction constant and n sub-variables corresponding to the individual variable.

Preferably, the method further comprises a method for establishing the prediction coefficient matrix comprising: retrieving a reference image set comprising a plurality of reference medical images; measuring a reference body composition index corresponding to each one of the reference medical images, and the reference body composition index comprises a reference muscle index or a reference fat index; extracting a reference individual variable corresponding to each one of the reference medical images, and the reference individual variable comprises n reference sub-variables; estimating m prediction coefficient sets by conducting a quantile regression analysis based on the reference body composition index and the reference individual variable, wherein any one of the prediction coefficient sets contains a prediction constant and a sub-prediction coefficient set corresponding thereto, and wherein the sub-prediction coefficient set contains n of the prediction coefficients and each one of the prediction coefficients corresponds one-to-one with each one of the reference sub-variables; and establishing the prediction coefficient matrix based on the prediction coefficient sets and the prediction quantile values.

Preferably, before the body age evaluated value is produced, the method further comprises: retrieving a median prediction constant and a median prediction coefficient set from the prediction coefficient matrix, and building a body age evaluation function, wherein the median prediction constant corresponds to the prediction constant of a median prediction quantile, and the median prediction coefficient set corresponds to the prediction coefficient set of a median prediction quantile; and inputting the body composition index and the individual variable to the body age evaluation function to compute the body age evaluated value corresponding thereto.

Preferably, the muscle index comprises a muscle mass index, a muscle labelling index, and a muscle area index; the muscle mass index comprises SMI (Skeletal Muscle Index), SMD (Skeletal Muscle Density), ImatA (Intramuscular Adipose Tissue Area), ImatD (Intramuscular Adipose Tissue Density), NamaA (Normal Attenuation Muscle Area), LamaA (Low Attenuation Muscle Area) or a combination thereof; the muscle labelling index comprises LWM (Lean Whole-body Mass), Total (Total Muscle Mass) or an assigned muscle group selected from a group consisting of lumbar vertebrae side muscle group, leg muscle group, chest muscle group, dorsal muscle group, ventral muscle group, biceps muscle group, triceps muscle group, and core muscle group; the muscle area index comprises VBA (Vertebral Body Area); the fat index comprises VatA (Visceral Adipose Tissue Area), VatD (Visceral Adipose Tissue Density), SatA (Subcutaneous Adipose Tissue Area), SatD (Subcutaneous Adipose Tissue Density) or any combination thereof; the comprehensive index comprises a muscle volume comprehensive index, a muscle quality comprehensive index, a muscle comprehensive index, a visceral fat index or a body comprehensive index; the n sub-variables of the individual variable is extracted from an individual information corresponding to the medical image, and comprises a gender variable, an age variable or a combination thereof.

In another aspect, the present invention provides a system for evaluating body composition, comprising: an imaging analysis module, configured to measure and output a body composition index based on a medical image, and to extract an individual variable corresponding to the medical image, wherein the body composition index comprises a muscle index, a fat index or a comprehensive index; and a prediction module, being signal-connected to the imaging analysis module, configured of a body composition prediction model, wherein the body composition index and the individual variable are input to the body composition prediction model so as to produce a body composition evaluation index comprising a body composition quantile value or a body age evaluated value.

Preferably, the prediction module is further configured of a body age evaluation unit, and body age evaluation unit retrieves a median prediction constant and a median prediction coefficient set from the prediction coefficient matrix, and builds a body age evaluation function, wherein the median prediction constant corresponds to the prediction constant of a median prediction quantile, and the median prediction coefficient set corresponds to the prediction coefficient set of a median prediction quantile value; the body age evaluation unit inputs the body composition index to the body age evaluation function so as to compute an undetermined body age variables and outputs the body age evaluated value corresponding thereto.

The method and system disclosed in the present invention provides means for rapid quantitative analysis and tracking of body composition based on medical images; it requires only one medical image of a to-be-tested subject, such as a ventral CT scanning image, to accomplish multiple types of body composition quantitative analysis including skeletal muscle, muscle fat or organ fat. A quantile value of the to-be-tested subject's body composition among a normal distributed group can also be automatically predicted, and body age thereof is further estimated. Such information about body composition can be basis for judgement of the to-be-tested subject as a healthy, sub-healthy or having risk of muscle loss-related disease, given same sex, age, weight or other physiological conditions.

### BRIEF DESCRIPTION OF THE DRAWINGS

FIG. 1A is a flow chart to illustrate a method provided in the first aspect of the present invention;

FIG. 1B is a matrix diagram to illustrate structure of body composition prediction model in the first aspect of the present invention;

FIG. 1C is a flow chart to illustrate a method for evaluating a body composition index in the first aspect of the present invention;

FIG. 1D is a CT scan image to exemplify muscle region, fat region and bone region;

FIG. 2 is a flow chart to illustrate a method provided in the second aspect of the present invention;

FIG. 3 is a flow chart to illustrate a method for establishing prediction coefficient matrix in the third aspect of the present invention;

FIGS. 4A to 4B are block diagrams to illustrate system structure and interconnection of the elements in the fourth and fifth aspects of the present invention;

FIGS. 5A to 5B are matrix diagram and curve plot to explain computational process of prediction coefficient matrix Total×SMI and unfolded curve on a two-dimensional plane in Example 2, respectively;

FIGS. 6A to 6B are matrix diagram and curve plot to explain computational process of prediction coefficient matrix LWM×SMI and unfolded curve on a two-dimensional plane in Example 3, respectively;

FIGS. 7A to 7B are matrix diagram and curve plot to explain computational process of prediction coefficient matrix PSM×SMI and unfolded curve on a two-dimensional plane in Example 3, respectively; and

FIGS. 8A to 8B are matrix diagram and curve plot to explain computational process of prediction coefficient matrix Total×muscle volume comprehensive index and unfolded curve on a two-dimensional plane in Example 12, respectively.

### DETAILED DESCRIPTION OF THE INVENTION

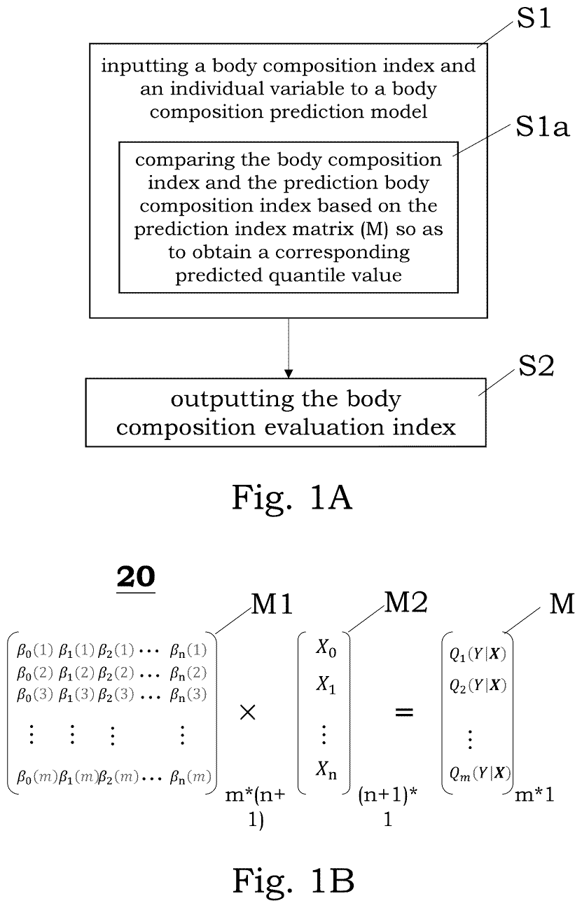

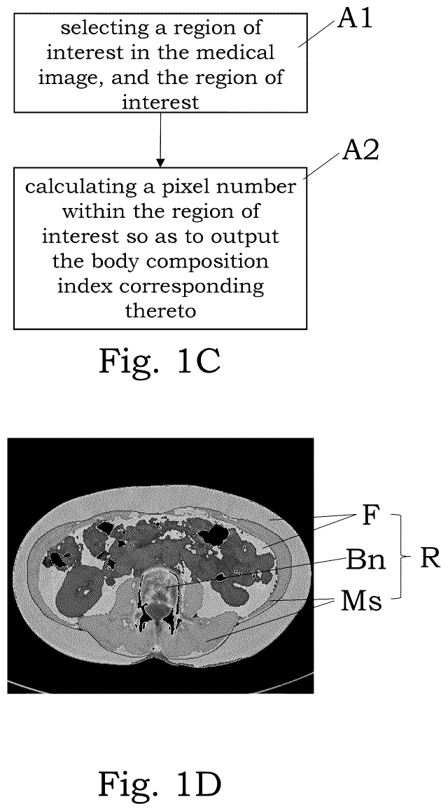

Please refer to FIG. 1A, the first aspect in the present invention is a method for evaluating body composition, comprising:
Step S1: inputting a body composition index and an individual variable to a body composition prediction model so as to produce a body composition evaluation index, wherein the body composition evaluation index comprises a body composition quantile value or a body age evaluated value, and wherein the body composition index is measured based on a medical image, and comprises a muscle index, a fat index or a comprehensive index, and the individual variable corresponds to an individual variable of the medical image; and
Step S2: outputting the body composition evaluation index.

In some embodiments, the body composition prediction model (20) accomplishes prediction of the body composition quantile based on a prediction index matrix (M); in detail, please refer to FIG. 1B, the method further comprises:

Step S1a: comparing the body composition index and the prediction body composition index based on the prediction index matrix (M) so as to obtain a corresponding predicted quantile value outputted to be the body composition quantile value.

FIG. 1B demonstrates the prediction index matrix (M), which is a matrix of dimension m*1, comprising m prediction body composition index. The prediction body composition index is represented by Q₁(Y|X) to Qₘ(Y|X), but not limited to this. Comprehensibly, the 1^{st} dimension in the matrix corresponds to the first quantile value; in other words, the body composition prediction model is defaulted to contain m prediction quantile values and a prediction index matrix so that the prediction body composition index corresponds one-to-one with the prediction quantile values. The quantile values are not limited to any one of positive integers between 1 to 100, and the quantile values can be any one of decimal values between 1 to 100, such as 1.0, 1.1, 1.2, 1.3 . . . 99.9 and 100.0, depending on user's quantile definition. In one example, m is any one of positive integers larger or equal to 1, and m can be any one of positive integers between 1 to 100.

In some preferred embodiments, please continue with FIG. 1B, the prediction index matrix (M) is derived from a multiplication of a prediction coefficient matrix (M1) and an individual variable matrix (M2), wherein: the prediction coefficient matrix (M1) is a matrix of dimension m*(n+1) comprising a prediction constant corresponding to each one of the prediction quantile values and a prediction coefficient set corresponding to each one of the prediction quantile values, and wherein the prediction coefficient set comprises n prediction coefficients, and wherein n is any one of positive integers larger or equal to 1. The individual variable matrix (M2) is a matrix of dimension (n+1)*1 comprising a prediction variable corresponding to the prediction constant and n sub-variables corresponding to the individual variable. To be specific, back in FIG. 1B, the prediction constant is denoted by β₀(1) to β₀(m), and the prediction coefficient corresponding to one of the prediction quantile values, such as the first quantile value, is denoted by β₁(1) to βₙ(1), and so on for other prediction coefficients. On the other hand, the prediction variable is denoted by X₀, and the sub-variable is denoted by X₁ to Xₙ. Comprehensibly, the prediction constant, prediction coefficient, prediction variable and sub-variable, as denoted by the function and the mathematical symbols, are solely to explain design of the prediction coefficient matrix (M1) and the individual variable matrix (M2), but not intended to limit their implementation.

In one more preferred embodiment, the prediction body composition index can be calculated by the following prediction function (I):

$${Q}_{q} ( Y \vert X ) = {β}_{0} ( q ) + {β}_{1} ( q ) {X}_{1} + {β}_{2} ( q ) {X}_{2} + ⋯ + {β}_{n} ( q ) {X}_{n} \qquad ( I )$$

wherein: Q_{q}(Y|X) presents the prediction body composition index; X presents the individual variable; Y presents a predicted value on condition of X; q is any one of positive integers lower than m; β₀(q) presents the prediction constant; the n prediction coefficients contain β₁(q) to βₙ(q); the n sub-variables contain X₁ to Xₙ.

Notably, compared with traditional regression analysis whose β₀, β₁, β₂, β₃ are constant parameters, among the n prediction coefficients β₀(q), β₁(q), β₂(q), β₃(q) . . . , with quantile q variating, various sets of β₀, β₁, β₂, β₃ . . . βₙ will be obtained. Namely, given changing prediction quantile values, such as a 1^{st} quantile (q is 1) and a 25^{th} quantile (q is 25), two different sets of prediction coefficients, β₀(1), β₁(1), β₂(1), β₃(1) . . . β₀(1) and β₀(25)·β₁(25), β₂(25), β₃(25) . . . βₙ(25), would be obtained, respectively. Therefore, based on variation in the quantity m of the prediction quantile values, the dimensions of the subsequently generated prediction coefficient matrix also change accordingly. For example, as illustrated in table 1a, when m is assigned as 100, a coefficient matrix of dimension 100*(n+1) is produced as the prediction coefficient matrix.

TABLE 1a

| | individual | individual | individual | individual | | individual |
|---|---|---|---|---|---|---|
| | variable 0 | variable 1 | variable 2 | variable 3 | | variable n |
| | prediction | prediction | prediction | prediction | | prediction |
| quantile | constant | constant | constant | constant | | constant |
| (q) | β₀(q) | β₁(q) | β₂(q) | β₃(q) | . . . | βₙ(q) |
| 1 | β₀(1) | β₁(1) | β₂(1) | β₃(1) | . . . | βₙ(1) |
| 2 | β₀(2) | β₁(2) | β₂(2) | β₃(2) | . . . | βₙ(2) |
| 3 | β₀(3) | β₁(3) | β₂(3) | β₃(3) | . . . | βₙ(3) |
| . | . | . | . | . | . . . | . |
| . | . | . | . | . | | . |
| . | . | . | . | . | | . |
| 100 | β₀(100) | β₁(100) | β₂(100) | β₃(100) | . . . | βₙ(100) |

In some embodiments, the medical image comprises any one of the following: CT image, fMRI image, X-ray image, ultrasound image, or pathological biopsy image, as long as the image is able to demonstrate a cross-sectional image of a specific subject's body. In some preferred embodiments, the cross-sectional image can be an image of body cross-section at any segment of the cervical spine, thoracic spine, lumbar spine, sacrum, or coccyx. Preferably, the medical image is an image of cross-section at lumbar spine, such as vertebral body L1, vertebral body L2, vertebral body L3, vertebral body L4 or vertebral body L5.

Furthermore, after capturing of the medical image, the body composition index can be measured based on various regions in the image of body cross-section. As shown in FIGS. 1C to 1D, a method for measurement comprises:
Step A1: selecting a region of interest (R) in the medical image, and the region of interest (R) comprise a muscle region (Ms), a bone region (Bn) or a fat region (F); and
Step A2: calculating a pixel number within the region of interest (R) so as to output the body composition index corresponding thereto.

In some exemplary embodiments, CT image in DICOM format can be obtained by pre-processing of the image of body cross-section with a Matlab-programmed semi-automatic image processing system such as Analytic Morphomics. Regions of interest are subsequently segmented by using a medical imaging analysis software in a manual or automatic manner. The region of interest (R) can be segmented into muscle region (Ms), bone region (Bn), or fat region (F) including regions of SAT (subcutaneous adipose tissue) and VAT (visceral adipose tissue). In one specific example, the medical imaging analysis software can be sliceOmatic, but not limited to this In some other examples, the regions of interest can be generated automatically by using FCN-based (fully convolutional networks-based) imaging segmentation techniques, which enables automatic selection of medical images.

In the aforementioned embodiments, the muscle index is obtained by calculation based on the muscle region (Ms). The muscle index refers to multiple types of parameters for assessing muscle mass and quality, and comprising a muscle mass index, a muscle labelling index or a muscle area index.

In one or various embodiments, the muscle mass index can be exemplified by SMI (Skeletal Muscle Index), SMD (Skeletal Muscle Density), ImatA (Intramuscular Adipose Tissue Area), ImatD (Intramuscular Adipose Tissue Density), NamaA (Normal Attenuation Muscle Area) or LamaA (Low Attenuation Muscle Area). In one preferred embodiment, analysis of muscle mass index can be performed by using SAS software or R software, but not limited to this.

Compared with the muscle mass index which indicates density or fat content of muscle, the muscle labelling index is adapted to underline a muscle mass benchmark or a specific muscle portion for measuring the muscle mass index. The muscle mass benchmark includes LWM (Lean Whole-body Mass) or Total (Total Muscle Mass). The specific muscle portion can be an assigned muscle group including lumbar vertebrae side muscle group, core muscle group, leg muscle group, chest muscle group, dorsal muscle group, ventral muscle group, biceps muscle group or triceps muscle group, and the muscle area index can take reference with VBA (Vertebral Body Area). More specifically, LWM refers to whole body mass without fat, and muscle, bone and water are all inclusive; Total refers to the entire muscle mass of the whole body.

In certain embodiments, the assigned muscle group is selected from a group consisting of PM (Psoas Muscle), PSM (Paraspinal Muscle), QLM (Quadratus Lumborum Muscle), RM (Rectus Muscle), (Transversus Abdominis Muscle), PFM (Pelvic Floor Muscles), MM (Multifidus Muscle), IOM (Internal Obliques Muscle), EOM (External Obliques Muscle) and LTM (Longissimus Thoracis Muscle).

In some other embodiments, when calculating the total muscle mass, a muscle area index can be further taken into consideration. The muscle area index can be Vertebra body area (VBA), wherein, with regard of different section of vertebral column, VBA comprises cervical vertebrae area (CVA), thoracic vertebrae area (TVA), lumbar vertebrae area (LVA) or sacrum area (SA); the aforementioned vertebral sections can be further subdivided according to anatomical definition, such as C1 to C7 cervical vertebrae, T1 to T12 thoracic vertebrae, L1 to L2 lumbar vertebrae, or S1 to S5 sacrum, but not limited to this.

In some embodiments, the fat index is derived from measurement based on the fat region (F), and comprises VatA (Visceral Adipose Tissue Area), VatD (Visceral Adipose Tissue Density), SatA (Subcutaneous Adipose Tissue Area), SatD (Subcutaneous Adipose Tissue Density) or any combination thereof. In concrete terms, VatA (Visceral Adipose Tissue Area) refers to a total area of the fat regions surrounding internal organs inside the abdominal cavity; VatD (Visceral Adipose Tissue Density) refers to density of the aforementioned fat surrounding the internal organs; SatA (Subcutaneous Adipose Tissue Area) refers to a total area of fat regions between epidermis and the muscle layer; SatD (Subcutaneous Adipose Tissue Density) refers to density of subcutaneous fat in the aforementioned total area of fat regions between epidermis and the muscle layer.

In some other embodiments, a bone index can be further derived from measurement based on the bone region (Bn), and comprises BV/TV (Bone Volume Fraction), TbN (Trabecular Number), TbTh (Trabecular Thickness) or TbSp (Trabecular Separation).

In yet some embodiments, the comprehensive index comprises a muscle volume comprehensive index, a muscle quality comprehensive index, a muscle comprehensive index, a visceral fat index or a body comprehensive index; to further elaborate, calculations of the comprehensive index involve the muscle index, the fat index, or the bone index as its key parameters. Table 1b provides several exemplary approaches to calculate the comprehensive index, but not limited to this.

TABLE 1b

| Comprehensive index | Approaches |
|---|---|
| muscle volume | SMI + SMD + VBA |
| comprehensive index | |
| muscle quality | NamaA + LamaA + ImatA |
| comprehensive index | |
| muscle comprehensive index | SMI + SMD + VBA + NamaA + |
| | LamaA + ImatA |
| internal fat index | VatA + SatA + NamaA + LamaA + |
| | ImatA |
| body comprehensive index | VatA + SatA + SMI + SMD + |
| | NamaA + LamaA + ImatA |

In one or more embodiments, the n sub-variables of the individual variable are extracted from an individual information corresponding to the medical image. As can be understood, the individual information refers to a variety of parameters including gender, age, body height, body weight, BMI, body fat rate, total muscle rate, blood fat, blood sugar or blood pressure. Any one of the sub-variables is presented in a numerical value. For instance, a male is presented in 1, while a female is presented in 0; similarly, when age is 20 years old, it is presented in 20; another instance is that body weight of 60 kilograms is presented in 60; for yet another instance, when body fat rate is 21%, it is presented in 21, and so on for other parameters.

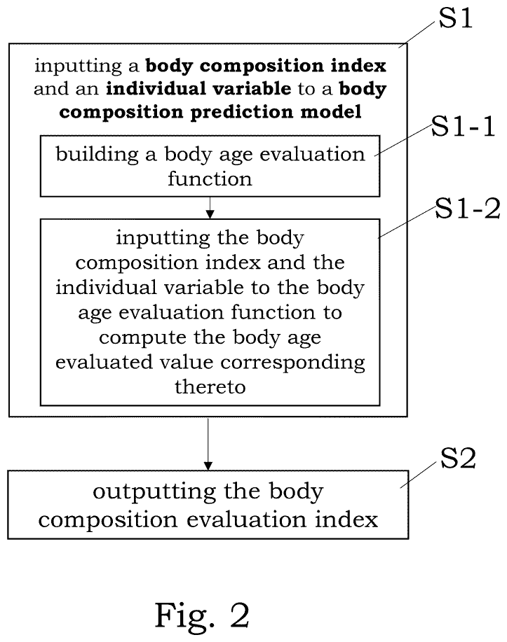

Shown in FIG. 2 is the second aspect of the present invention providing a method for evaluating body composition, and steps, processing logic of the method are overall the same as the method in the first aspect. Thus, details of the method are not further illustrated hereinafter, but the body composition prediction model outputs a body age evaluated value to be the body composition evaluation index, wherein, before the body composition index and the individual variable are input, the method further comprises:
Step S1-1: building a body age evaluation function; and
Step S1-2: inputting the body composition index and the individual variable to the body age evaluation function to compute the body age evaluated value corresponding thereto.

In particular embodiments, the building body age evaluation function step comprises: retrieving a median prediction constant and a median prediction coefficient set from the prediction coefficient matrix so as to build the body age evaluation function, wherein the median prediction constant corresponds to the prediction constant of a median prediction quantile, and the median prediction coefficient set corresponds to the prediction coefficient set of a median prediction quantile; concretely, the body age evaluation function follows a function (II):

$${Q}_{age} = {β}_{0} ( {q}_{me} ) + {β}_{1} ( {q}_{me} ) {X}_{me 1} + {β}_{2} ( {q}_{me} ) {X}_{me 2} + ⋯ + {β}_{n} ( {q}_{me} ) {X}_{men} \qquad ( II )$$

wherein, Q_{age} represents the body composition index, q represents one of any positive integers smaller than m, β₀(qₘₑ) represents the prediction constant, the median prediction coefficient set comprises β₁(qₘₑ) to βₙ(qₘₑ), the Xₘₑ₁ represents a gender variable, and the Xₘₑ₂ to Xₘₑₙ represent body age variables; and

In specific embodiments, the median prediction constant is the prediction constant of the 50^{th} quantile, and the median prediction coefficient set is a prediction coefficient set corresponding to the 50^{th} quantile. The body composition index is the comprehensive index, such as muscle volume comprehensive index, muscle quality comprehensive index, muscle comprehensive index, internal fat index or body comprehensive index, and the individual variable is the gender variable.

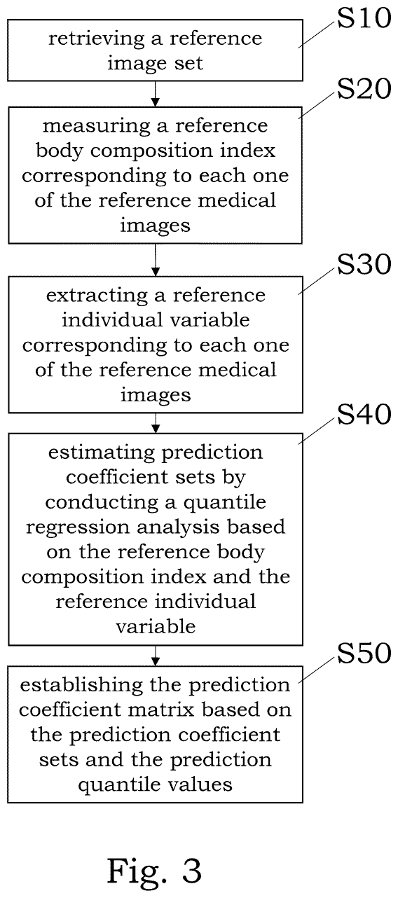

With reference to FIG. 3, the third aspect of the present invention is to provide a technical solution that is applicable to the first aspect and the second aspect, wherein a method for establishing the prediction coefficient matrix is provided herein, comprising:
Step S10: retrieving a reference image set comprising a plurality of reference medical images;
Step S20: measuring a reference body composition index corresponding to each one of the reference medical images, and the reference body composition index comprises a reference muscle index or a reference fat index;
Step S30: extracting a reference individual variable corresponding to each one of the reference medical images, and the reference individual variable comprises n reference sub-variables;
Step S40: estimating m prediction coefficient sets by conducting a quantile regression analysis based on the reference body composition index and the reference individual variable, wherein any one of the prediction coefficient sets contains a prediction constant and a sub-prediction coefficient set corresponding thereto, and wherein the sub-prediction coefficient set contains n of the prediction coefficients and each one of the prediction coefficients corresponds one-to-one with each one of the reference sub-variables; and
Step S50: establishing the prediction coefficient matrix based on the prediction coefficient sets and the prediction quantile values.

In various embodiments, nonparametric quantile regression analysis is applied. As illustrated in the first aspect of the present invention, the aforesaid prediction function (I) is adapted to calculate the prediction body composition index, namely Q_{q}(Y|X), and the prediction body composition index corresponds to each one of the quantile values q. The purpose of using quantile regression analysis to analyze the reference body composition index and the reference sub-variables lies in estimation of prediction constant β₀(q) and prediction coefficient β₁(q) to βₙ(q) corresponding to the quantile value q. Proper approaches for such estimation include minimization of a loss function. As being distinguished from Ordinary least square (OLS), minimization of a loss function is adapted to minimize residual between the output value from the prediction function (I) and the actual value on condition of a given quantile value q, while OLS is adapted to minimize residual sum of squares.

Hereinafter, a process for parametric estimation of the quantile regression analysis is further illustrated with a specific example:

A loss function is designed as the following function (i), and, with the loss function, a sum of absolute residuals having minimized weighing can be calculated. The weighing includes the prediction constant β₀(q) and prediction coefficient β₁(q) to βₙ(q), depending on quantile value q:

$${\sum }_{i = 1}^{n} {ρ}_{( q )} ( {Y}_{i} - ( {β}_{0} ( q ) + {β}_{1} ( q ) {X}_{1 i} + {β}_{2} ( q ) {X}_{2 i} ⋯ {⋯β}_{n} ( q ) {X}_{ni} ) ) \qquad ( i )$$

ρ_{(q)} represents a quantile loss function, and ρ_{(q)}(u)=u[q−I(u<0)], q∈(0,1), wherein: I represents an index function, Xₙᵢ represents an explanatory variables (a.k.a. the reference sub-variable), Yᵢ represents a dependent variable of actual observation (a.k.a. the reference body composition index), and u represents the residual value (a.k.a. the residual between actual observed value and the predicted value estimated by the prediction function (I)).

Accordingly, based on the sum of absolute residuals with weighing, during the building process of the prediction coefficient matrix, the weight assigned to each quantile value q would be determined depending on whether the weighted residuals derived from the quantile loss function ρ_{(q)}(u)=u[q−I(u<0)] are positive or negative. In other words, during minimization process of the residual, the prediction constant β₀(q) and prediction coefficient β₁(q) to βₙ(q) corresponding to the given quantile value q are tuned stepwise until the residual between the prediction value output by the prediction function (I) and the actual observed value is minimized.

In various embodiments, the minimized value within the quantile regression analysis can be solved by a normal numerical operation, such as Newton's method. In practical uses, the computation can be performed with functions available in statistics software, such as quantreg of R software, scikit-learn or statsmodels of Python, SAS, SPSS, Stata, MATLAB or Excel.

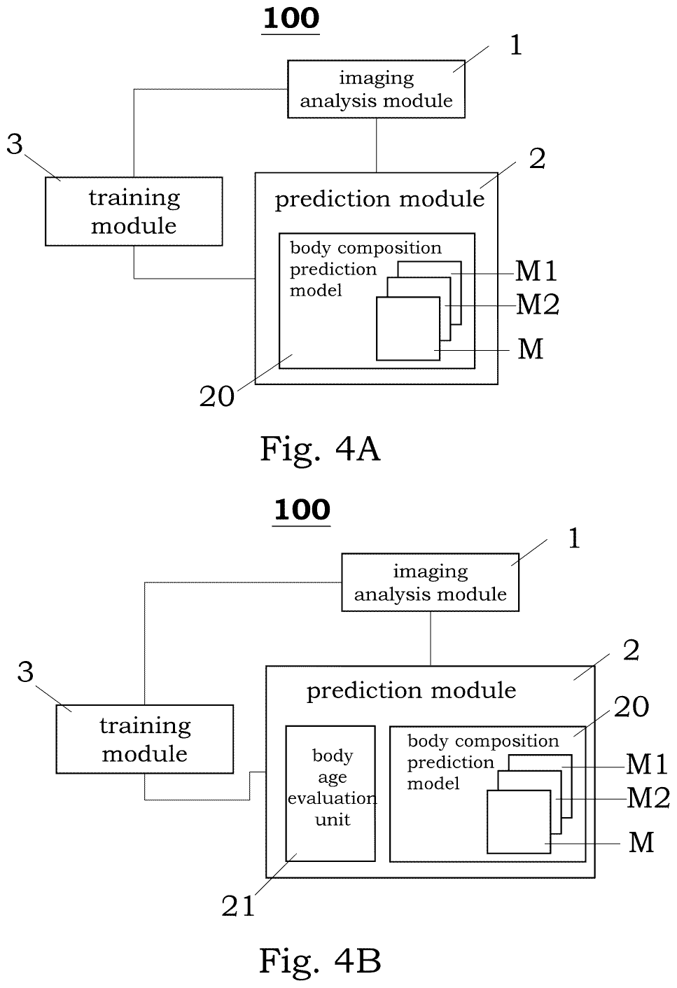

Please refer to FIG. 4A, the fourth aspect of the present invention provides a system (100) for evaluating body composition, comprising:
an imaging analysis module (1), configured to measure and output a body composition index based on a medical image, and to extract an individual variable corresponding to the medical image, wherein the body composition index comprises a muscle index, a fat index or a comprehensive index; and
a prediction module (2), being signal-connected to the imaging analysis module (1) and configured of a body composition prediction model, wherein the body composition index and the individual variable are input to the body composition prediction model (20) so as to produce a body composition evaluation index, wherein the body composition evaluation index comprises a body composition quantile value.

In the fourth aspect of the present invention, the body composition prediction model (20) comprises a prediction coefficient matrix (M1), an individual variable matrix (M2) and a prediction index matrix (M), wherein coefficient configuration, function design and building methods of the individual variable matrix (M2) and the prediction index matrix (M) have been disclosed in the first aspect of the present invention and will not be further elaborated upon here.

In various embodiments, the system (100) further comprises a training module (3), being signal-connected to the imaging analysis module (1) and the prediction module (2) and configured to establish the prediction coefficient matrix (M1), wherein: the imaging analysis module (1) obtains a reference body composition index and a reference individual variable based on any one of reference medical images of a reference image set, wherein the reference body composition index comprises a reference muscle index or a reference fat index, and the reference individual variable comprises n reference sub-variables; the training module (3) conducts a quantile regression analysis based on the reference body composition index and the reference individual variable so as to estimate m prediction coefficient sets, and wherein any one of the prediction coefficient sets comprises a prediction constant and a sub-prediction coefficient set corresponding thereto, wherein the sub-prediction coefficient set comprises n of the prediction coefficients, and each of the prediction coefficients corresponds one-to-one with each of the reference sub-variables; and the training module (3) establishes the prediction coefficient matrix (M1) based on the prediction coefficient sets and the prediction quantile values, and output the prediction coefficient matrix (M1) to the prediction module (2).

In the fourth aspect of the present invention, the muscle index, fat index, comprehensive index, individual variable, approaches of quantile regression analysis and medical image segmentation have been detailed in the first aspect of the present invention and will not be further elaborated upon here.

Please refer to FIG. 4B, the fifth aspect of the present invention provides a system (100) for evaluating body composition, and elements, connections among the elements and processing logic thereof are overall the same as the system (100) in the fourth aspect of the present invention and will not be further discussed in detail upon here, but the prediction module (2) is further configured of a body age evaluation unit (21), and body age evaluation unit is configured to build a body evaluation function so that a body age evaluated value can be derived therefrom according to the body composition index and the individual variable.

In particular embodiments, the body age evaluation unit (21) retrieves a median prediction constant and a median prediction coefficient set from the prediction coefficient matrix (M1), and builds a body age evaluation function, wherein the median prediction constant corresponds to the prediction constant of a median prediction quantile, and the median prediction coefficient set corresponds to the prediction coefficient set of a median prediction quantile, wherein the body age evaluation function and body composition index have been discussed in detail in the second aspect of the present invention, and no further elaboration will be provided here.

### Example 1

Among 5302 abdominal CT scan images provided by National Chung Kung University Hospital, those were diagnosed of diseases, such as cardiovascular diseases, sarcopenia, or metabolic syndromes including hypertension or hyperlipemia, were excluded. After the aforesaid cases sorting, 4603 images captured from healthy group of age between 20 to 80 years old remained.

The aforementioned images were input to the system (100) for evaluating body composition, and were segmented by the imaging analysis module (1). The regions of interest included a muscle region and a fat region, and a muscle index and a fat index were measured based on these regions of interest. When measurement of the muscle index was conducted, the muscle mass benchmark and specific muscle portions were also considered. Particularly, muscle indices including SMI, SMD, ImatA, ImatD were measured in regions assigned of muscle labelling indices LWM, PM, PSM, QLM, RM, Total. On the other hand, the fat indices including VatA, VatD, SatA, SatD were also measured.

Approaches for measurement of muscle mass and fat mass are based on “Method for measuring muscle mass” as disclosed in Taiwan Pat. No 1797976, and approaches for image segmentation are based on “Image segmentation method and electronic device, recording medium and computer program product using the same” in Taiwan Pat. No 1797672. Thus, further details are omitted here.

Subsequently, the imaging analysis module (1) further extracted information including gender and age of the individual corresponding to each of the CT scan images, and the extracted information were submitted as reference sub-variables. The reference sub-variables were combined with the muscle indices and fat indices, as retrieved in the previous procedure, to be reference body composition indices. The reference body composition indices were then input to the training module (3) for quantile regression analysis.

In Example 1, the reference sub-variables were specified as gender, age and square of age, and a prediction function (II) was designed as the following:

$${Q}_{q} ( Y \vert X ) = {β}_{0} ( q ) + {β}_{1} ( q ) {X}_{1} + {β}_{2} ( q ) {X}_{2} + {β}_{3} ( q ) {X}_{3} \qquad ( II )$$

Wherein: q represented quantile value; X₁ represented gender, and input was to be 1 if it referred to male, or input was to be 0 if it referred to female; X₂ represented age; X₃ represented square of age (age²).

When the training module (3) performed quantile regression analysis, the prediction constant β₀(q) and the prediction coefficients β₁(q), β₂(q), β₃(q) corresponding to each one of the quantile values were estimated by an approach of minimizing sum of residuals. With a given quantile value q, the prediction constant β₀(q) corresponded to an intercept of the model, and prediction coefficients β₁(q), β₂(q), β₃(q) were regression coefficients of the reference sub-variables: gender, age and square of age. After parameter tuning by regression analysis, the residual between the predicted value Q_{q}(Y|X) and the actual measured value was minimized.

In another aspect, considering that 4 different muscle mass indices corresponded to one single muscle labelling index, and with regard of 6 different muscle mass benchmarks and specific muscle portions in Example 1, 24 distinct prediction coefficient matrixes (M1) were established accordingly. These matrixes included the followings: LWM×SMI [symbol] LWM×SMD [symbol] LWM×ImatA [symbol] LWM×ImatD [symbol] PM×SMI [symbol] PM×SMD [symbol] PM×ImatA [symbol] PM×ImatD [symbol] PSM×SMI [symbol] PSM×SMD [symbol] PSM×ImatA [symbol] PSM×ImatD [symbol] QLM×SMI [symbol] QLM×SMD [symbol] QLM×ImatA [symbol] QLM×ImatD [symbol] RM×SMI [symbol] RM×SMD [symbol] RM×ImatA [symbol] RM×ImatD [symbol] Total×SMI [symbol] Total×SMD [symbol] Total×ImatA [symbol] Total×ImatD. At mean time, when considering another 4 different fat indices, 4 distinct prediction coefficient matrixes (M1) were also established.

In Example 1, each one of the prediction coefficient matrixes (M1) was defaulted to contain 100 quantile values including 1%, 2% . . . to 100%. In combination of the prediction constant and 3 prediction coefficients, a matrix of dimension 100*4 was built, and eventually 28 distinct prediction coefficient matrixes (M1) corresponding to different muscle indices and fat indices were established.

For instance, the muscle mass index for measurement was specified as SMI on condition of the muscle mass benchmark Total, and specific contents of the prediction coefficient matrix (M1) can be found in Table 2. In Table 2, listed along the horizontal array were prediction coefficients corresponding to specific quantile values. Taking 1% for example, its prediction constant was 28.211, gender prediction coefficient was 8.404, age prediction coefficient was 0.029 and coefficient of square of age was 0.00103.

TABLE 2

| Quantile | Intercept | Gender | Age | Age² |
|---|---|---|---|---|
| (q) | β₀(q) | β₁(q) | β₂(q) | β₃(q) |
| 1% | 28.211 | 8.404 | 0.029 | −0.00103 |
| 2% | 27.501 | 9.006 | 0.118 | −0.00191 |
| 3% | 26.694 | 9.120 | 0.196 | −0.00266 |
| . | . | . | . | . |
| . | . | . | . | . |
| . | . | . | . | . |
| 100% | 99.311 | 9.906 | −1.348 | 0.01276 |

For another instance, the muscle mass index for measurement was specified as SMI on condition of the muscle mass benchmark LWM, and specific contents of the prediction coefficient matrix (M1) can be found in Table 3. In Table 3, listed along the horizontal axis were prediction coefficients corresponding to specific quantile values. Taking 1% for example, its prediction constant was 8.815, gender prediction coefficient was 2.499, age prediction coefficient was 0.030 and coefficient of square of age was 0.000443.

TABLE 3

| Quantile | Intercept | Gender | Age | Age² |
|---|---|---|---|---|
| (q) | β₀(q) | β₁(q) | β₂(q) | β₃(q) |
| 1% | 8.815 | 2.499 | 0.030 | −0.000443 |
| 2% | 10.523 | 2.847 | −0.029 | 0.000131 |
| 3% | 10.660 | 3.178 | −0.027 | 0.000143 |
| . | . | . | . | . |
| . | . | . | . | . |
| . | . | . | . | . |
| 100% | 32.184 | 4.584 | −0.174 | 0.000724 |

### Example 2

A L3 lumbar vertebral CT scan image was input to the prediction system (100) trained in Example 1 for subsequent analysis, and the muscle mass index for measurement was specified as SMI on condition of the muscle mass benchmark Total. The actual measured value of SMI by using the imaging analysis module (1) was 40, and the extracted individual information corresponding thereto was: female, 50 years old. Input parameters were listed in Table 4.

TABLE 4

| Input parameter | Parameter Value |
|---|---|
| Gender | female |
| Age | 50 |
| Actual measured value | 40 |
| (Total × SMI) | |
| Muscle mass index | SMI |
| Muscle labelling index | Total |

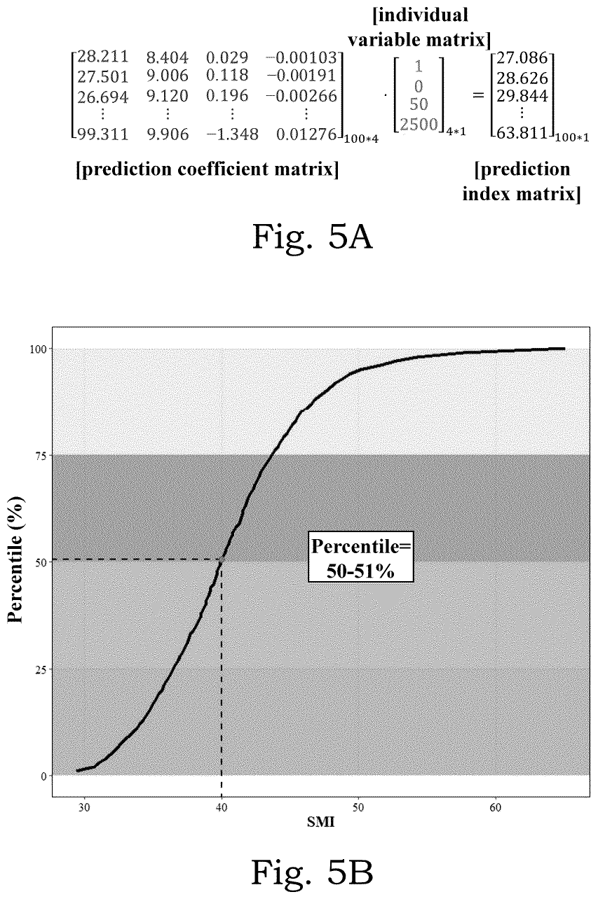

According to the input parameters in Table 4, the prediction module (2) further produced an individual variable matrix (M2). As shown in FIG. 5A, parameter values from the 1^{st} to the 4^{th} dimensions in the individual variable matrix (M2) were 1 (prediction variable), 0 (female), 50 (age) and 2500 (Age²). The prediction index matrix (M) was derived from a multiplication of the individual variable matrix (M2) and the prediction coefficient matrix (M1), and a curve of the prediction body composition indices from the 1^{st} to the 100^{th} dimensions corresponding to their percentiles and SMI values was plotted as shown in FIG. 5B. The curve was used to predict percentile of this female's SMI index among the health group, and it fell among 50 to 51%. Of course, the sequence in prediction index matrix (M) could also be unfolded and the closest percentile could be located by machine-based comparison in Example 2.

### Example 3

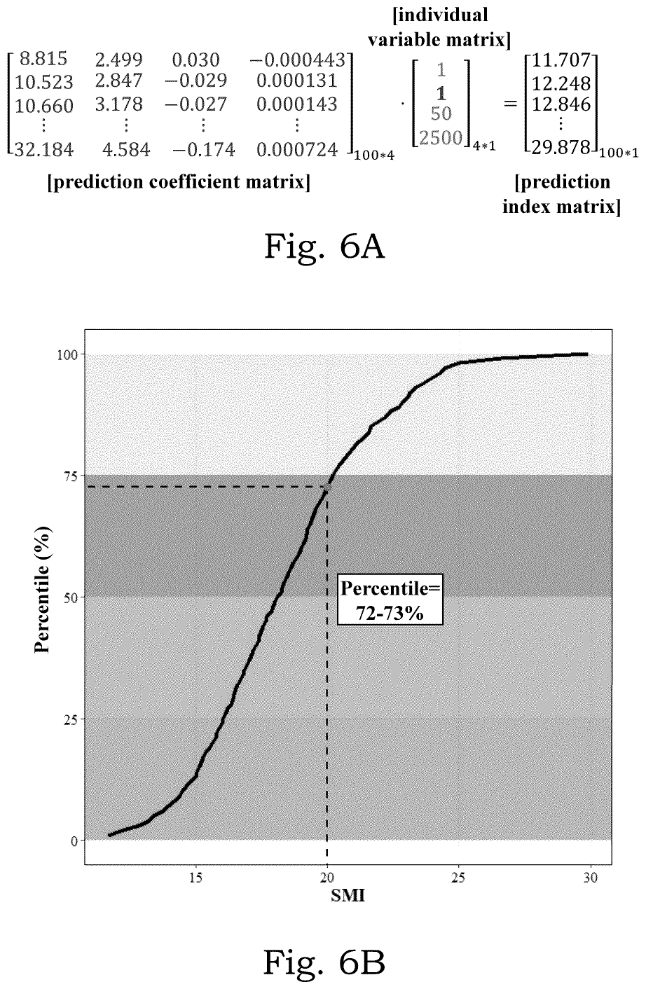

With minor alteration of Example 2, in Example 3, a certain sample was collected and input to the system (100), and parameters were listed in Table 5. The sample was a CT scan image of body cross-section at L3 lumbar vertebrae. Actual measured value of SMI on condition of LWM was 20, and individual information corresponding thereto was: male, 50 years old. Accordingly, the prediction module (2) further produced an individual variable matrix (M2). As shown in FIG. 6A, parameter values from the 1^{st} to the 4^{th} dimensions in the individual variable matrix (M2) were 1 (prediction variable), 1 (male), 50 (age) and 2500 (Age²). The prediction index matrix (M) was derived from a multiplication of the individual variable matrix (M2) and the prediction coefficient matrix (M1). As shown in FIG. 6B, a curve was plotted by unfolding the prediction index matrix (M). The predicted percentile fell among 72 to 73% by machine-based comparison.

TABLE 5

| Input parameter | Parameter Value |
|---|---|
| Gender | male |
| Age | 50 |
| Actual measured value | 20 |
| (LWM × SMI) | |
| Muscle mass index | SMI |
| Muscle labelling index | LWM |

### Example 4

In example 4, approaches to establish the prediction model were conducted with the system (100) as described in Example 1, but the reference sub-variables were substituted by gender, age and body weight, and the prediction function (Ia) was designed as following:

$${Q}_{q} ( Y \vert X ) = {β}_{0} ( q ) + {β}_{1} ( q ) {X}_{1} + {β}_{2} ( q ) {X}_{2} + {β}_{3} ( q ) {X}_{3}^{′} \qquad ( Ia )$$

Wherein: q represented quantile value; X₁ represented gender, and input was to be 1 if it referred to male, or input was to be 0 if it referred to female; X₂ represented age; X₃′ represented body weight.

Accordingly, the training module (3) conducted quantile regression analysis and built a prediction coefficient matrix (M1) of SMI on condition of PSM, and specific contents of this PSM×SMI prediction coefficient matrix (M1) can be found in Table 6. In Table 6, listed along the horizontal array were prediction coefficients corresponding to specific quantile values. Taking 1% for example, its prediction constant was 10.189, gender prediction coefficient was 2.390, age prediction coefficient was −0.043 and coefficient of body weight was 0.018.

TABLE 6

| | | | | Body |
|---|---|---|---|---|
| Percentile | Intercept | Gender | Age | weight |
| (q) | β₀(q) | β₁(q) | β₂(q) | β₃(q) |
| 1% | 10.189 | 2.390 | −0.043 | 0.018 |
| 2% | 9.877 | 2.448 | −0.019 | 0.021 |
| 3% | 8.675 | 1.917 | −0.016 | 0.049 |
| . | . | . | . | . |
| . | . | . | . | . |
| . | . | . | . | . |
| 100% | 4.339 | −1.588 | 0.041 | 0.334 |

### Example 5

A sample image of a male was input to the system (100) trained in Example 4, and input parameters were listed in Table 7. Similarly, the sample image was a CT scan image of body cross-section at L3 lumbar vertebrae. Actual measured value of SMI on condition of PSM was 15 and individual information corresponding thereto was: male, 50 years old, body weight 80 kilograms.

TABLE 6

| Input parameter | Parameter Value |
|---|---|
| Gender | male |
| Age | 50 |
| Body weight | 80 |
| Actual measured value | 15 |
| (PSM × SMI) | |
| Muscle mass index | SMI |
| Muscle labelling index | PSM |

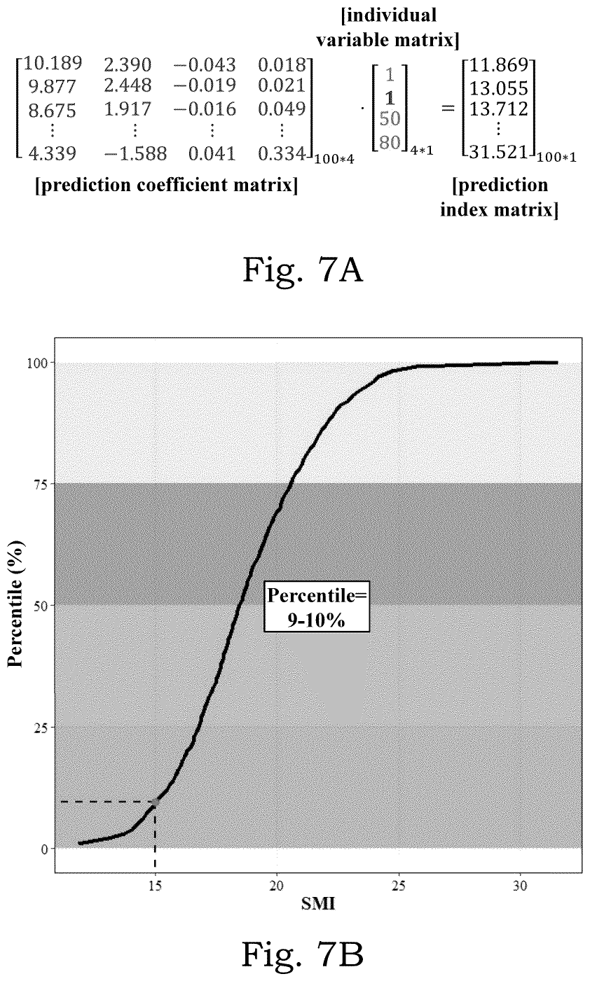

Accordingly, the prediction module (2) further produced an individual variable matrix (M2) as shown in FIG. 7A. Parameter values from the 1^{st} to the 4^{th} dimensions in the individual variable matrix (M2) were 1 (prediction variable), 1 (male), 50 (age) and 80 (body weight). The prediction index matrix (M) was derived from a multiplication of the individual variable matrix (M2) and the prediction coefficient matrix (M1). As shown in FIG. 7B, a curve was plotted by unfolding the prediction index matrix (M), and the predicted percentile fell among 9 to 10% by machine-based comparison.

### Example 6

With minor alteration of Example 1, muscle indices, fat indices and comprehensive indices were measured based on the regions of interest after image segmentation. Particularly, muscle indices including SMI, SMD, ImatA, ImatD, LamaA, NamaA were measured according to muscle labelling indices LWM, PM, PSM, QLM, RM, Total. On the other hand, the fat indices included VatA, VatD, SatA, SatD. The comprehensive indices were derived from the muscle indices and the fat indices following the approaches listed in Table 8.

TABLE 8

| comprehensive index | approaches |
|---|---|
| muscle volume | SMI + SMD + VBA |
| comprehensive index | |
| muscle quality | NamaA + LamaA + ImatA |
| comprehensive index | |
| muscle comprehensive index | SMI + SMD + VBA + NamaA + |
| | LamaA + ImatA |
| internal fat index | VatA + SatA + NamaA + LamaA + |
| | ImatA |
| body comprehensive index | VatA + SatA + SMI + SMD + |
| | NamaA + LamaA + ImatA |

Subsequently, the imaging analysis module (1) extracted information including gender and age of the individual corresponding to each of the CT scan images, and the extracted information were submitted as reference sub-variables. The reference sub-variables were combined with the muscle indices, the fat indices and the comprehensive indices, as retrieved in the previous procedure, to be reference body composition indices. The reference body composition indices were then input to the training module (3) for quantile regression analysis. Parameter settings of the reference sub-variables and regression analysis, as well as steps and processing logic thereof, were the same as described in Example 1, and details were omitted here.

Since there were 3 additional muscle comprehensive indices in the prediction coefficient matrix (M1), 18 distinct prediction coefficient matrixes (M1) were further established considering a one-to-one match of each muscle comprehensive index to corresponding muscle labelling index. The newly created prediction coefficient matrixes (M1) included: LWM×muscle volume comprehensive index, LWM×muscle quality comprehensive index, LWM×muscle comprehensive index, PM×muscle volume comprehensive index, PM×muscle quality comprehensive index, PM×muscle comprehensive index [symbol] PSM×muscle volume comprehensive index, PSM×muscle quality comprehensive index, PSM×muscle comprehensive index, QLM×muscle volume comprehensive index, QLM×muscle quality comprehensive index, QLM×muscle comprehensive index, RM×muscle volume comprehensive index, RM×muscle quality comprehensive index, RM×muscle comprehensive index, Total×muscle volume comprehensive index, Total×muscle comprehensive index, Total×muscle comprehensive index. Additionally, 6 distinct prediction coefficient matrixes were established according to internal fat indices and body comprehensive indices. Thus, 30 more prediction coefficient matrixes were created.

For instance, the muscle volume comprehensive indices were measured on condition of the muscle mass benchmark Total, and specific contents of the prediction coefficient matrix (M1) can be found in Table 9. In Table 9, listed along the horizontal array were prediction coefficients corresponding to specific quantile values. Taking 50% for example, its prediction constant was 91.145, gender prediction coefficient was 20.956, age prediction coefficient was 0.161 and coefficient of square of age was −0.00519. It should be noted that some of the prediction constants and the prediction coefficients were presented only up to the third decimal place in order for conciseness. In the subsequent Examples 7 to 12, all prediction constants and prediction coefficients involved in the body composition prediction

TABLE 9

| Percentile | Intercept | gender | Age | Age² |
|---|---|---|---|---|
| (q) | β₀(q) | β₁(q) | β₂(q) | β₃(q) |
| 1% | 83.993 | 16.136 | −0.224 | −0.00277 |
| 2% | 82.041 | 16.557 | −0.033 | −0.00465 |
| 3% | 81.497 | 17.642 | 0.034 | −0.00515 |
| . | . | . | . | . |
| . | . | . | . | . |
| . | . | . | . | . |
| 50% | 91.145 | 20.956 | 0.161 | −0.00519 |
| . | . | . | . | . |
| . | . | . | . | . |
| . | . | . | . | . |
| 100% | 173.582 | 25.117 | −1.650 | 0.00854 |

### Example 7

A sample image of a female was input to the system (100) trained in Example 6, and input parameters were listed in Table 10. The sample image was a CT scan image of body cross-section at L3 lumbar vertebrae. Actual measured values of SMI and SMD on condition of Total were 47.710824 and 33.6424240, respectively, and muscle area index VBA was 11.963267. The individual information corresponding thereto was: female, 50 years old. Furthermore, the imaging analysis module (1) summed up SMI, SMD and VBA and output 87.298331 as the muscle volume comprehensive index.

TABLE 10

| Input parameter | Parameter Value |
|---|---|
| Gender | female |
| Age | 50 |
| Muscle mass indices | SMI, SMD |
| muscle area index | VBA |
| Muscle labelling index | Total |
| Actual measured value | 41.710824 |
| (Total × SMI) | |
| Actual measured value | 33.624240 |
| (Total × SMD) | |
| Actual measured value | 11.963267 |
| (Total × VBA) | |
| muscle volume | 87.298331 |
| comprehensive index | |

Accordingly, the prediction module (2) built a body age evaluation function based on prediction coefficients corresponding to the 50th quantile value, and the function was presented in the following equation (IIa):

$${Q}_{age} = 91.145284441 + 20.956199309 {X}_{me 1} + 0.160548843 {X}_{me 2} - 0.005185815 {X}_{me 3} \qquad ( IIa )$$

Wherein: q represented quantile value; Xₘₑ₁ represented gender, and input was to be 1 if it referred to male, or input was to be 0 if it referred to female; Xₘₑ₂ represented age; Xₘₑ₃ represented square of age (age²).

Next, the prediction module (2) further substituted the muscle volume comprehensive index 87.298331 into Q_{age}, and the gender variable 0 into Xₘₑ₁, so as to solve a quadratic equation and obtained 46.807570 as the body age variable. The body age variable was then output as the body age evaluated value of this female. Compared with her actual age of 50 years old, the body age evaluated value was lower, which indicates that body composition of this female appears younger than her actual age in terms of muscle volume comprehensive index.

### Example 8

With minor alteration of Example 7, the actual measured values of muscle mass indices on condition of Total were: 64.215726 of NamaA, 35.250160 of LamaA and 12.032818 of ImatA, and individual information corresponding thereto was: female, 50 years old. Furthermore, the imaging analysis module (1) summed up NamaA, LamaA and ImatA and output 111.498704 as the muscle quality comprehensive index.

TABLE 11

| Input parameter | Parameter Value |
|---|---|
| Gender | female |
| Age | 50 |
| Muscle mass indices | NamaA, LamaA, ImatA |
| muscle area index | N/A |
| Muscle labelling index | Total |
| Actual measured value | 64.215726 |
| (Total × NamaA) | |
| Actual measured value | 35.250160 |
| (Total × LamaA) | |
| Actual measured value | 12.032818 |
| (Total × ImatA) | |
| muscle quality | 111.498704 |
| comprehensive index | |

Accordingly, the prediction module (2) built a body age evaluation function based on prediction coefficients corresponding to the 50^{th} quantile value of the muscle quality comprehensive index, and the function was presented in the following equation (IIb):

$${Q}_{age} = 94.387942997 + 55.858816492 {X}_{me 1} + 0.931984349 {X}_{me 2} - 0.012318074 {X}_{me 3} \qquad ( IIb )$$

Wherein: q represented quantile value; Xₘₑ₁ represented gender, and input was to be 1 if it referred to male, or input was to be 0 if it referred to female; Xₘₑ₂ represented age; Xₘₑ₃ represented square of age (age²).

Next, the prediction module (2) further substituted the muscle quality comprehensive index 111.498704 into Q_{age}, and the gender variable 0 into Xₘₑ₁, so as to solve a quadratic equation and obtained a body age variable 44.312841, which indicates that body composition of this female also appears younger than her actual age in terms of muscle quality comprehensive index.

### Example 9

With minor alteration of Example 8, the imaging analysis module (1) summed up muscle volume comprehensive index and muscle quality comprehensive index, and 198.797035 was output as the numerical value of a muscle comprehensive index.

The prediction module (2) built a body age evaluation function based on prediction coefficients corresponding to the 50^{th} quantile value of the muscle comprehensive index, and the function was presented in the following equation (IIc):

$${Q}_{age} = 188.35084977 + 76.564944432 {X}_{me 1} + 0.974711081 {X}_{me 2} - 0.016547935 {X}_{me 3} \qquad ( IIc )$$

Wherein: q represented quantile value; Xₘₑ₁ represented gender, and input was to be 1 if it referred to male, or input was to be 0 if it referred to female; Xₘₑ₂ represented age; Xₘₑ₃ represented square of age (age²).

Referring to the equation (IIc), the prediction module (2) substituted the muscle comprehensive index 198.797035 into Q_{age}, and the gender variable 0 into Xₘₑ₁, so as to solve a quadratic equation and obtained body age variable 44.816733, which indicates that body composition of this female also appears younger than her actual age in terms of muscle comprehensive index.

### Example 10

With minor alteration of Example 8, the imaging analysis module (1) summed up muscle quality comprehensive index and fat indices VatA and SatA, and 283.349233 was output as an internal fat comprehensive index.

The prediction module (2) built a body age evaluation function based on prediction coefficients corresponding to the 50^{th} quantile value of the muscle comprehensive index, and the function was presented in the following equation (IId):

$${Q}_{age} = 85.347336747 + 104.21228353 {X}_{me 1} + 7.806981552 {X}_{me 2} - 0.064961019 {X}_{me 3} \qquad ( IId )$$

Wherein: q represented quantile value; Xₘₑ₁ represented gender, and input was to be 1 if it referred to male, or input was to be 0 if it referred to female; Xₘₑ₂ represented age; Xₘₑ₃ represented square of age (age²).

Referring to the equation (IId), the prediction module (2) substituted the internal fat comprehensive index 283.349233 into Q_{age}, and the gender variable 0 into Xₘₑ₁, so as to solve a quadratic equation and obtained a body age variable 83.812430, which indicates that body composition of this female appears older than her actual age in terms of internal fat comprehensive index.

### Example 11

With minor alteration of Example 10, the imaging analysis module (1) summed up internal fat comprehensive index and muscle indices SMI and SMD, and 358.684297 was output as a body composition comprehensive index.

The prediction module (2) built a body age evaluation function based on prediction coefficients corresponding to the 50^{th} quantile value of the body composition comprehensive index, and the function was presented in the following equation (IIe):

$${Q}_{age} = 162.946879359 + 124.414335228 {X}_{me 1} + 8.167438545 {X}_{me 2} - 0.074323995 {X}_{me 3} \qquad ( IIe )$$

Wherein: q represented quantile value; Xₘₑ₁ represented gender, and input was to be 1 if it referred to male, or input was to be 0 if it referred to female; Xₘₑ₂ represented age; Xₘₑ₃ represented square of age (age²).

Referring to the equation (IIe), the prediction module (2) substituted the body composition comprehensive index 358.684297 into Q_{age}, and the gender variable 0 into Xₘₑ₁, so as to solve a quadratic equation and obtained a body age variable 74.575537, which indicates that body composition of this female also appears older than her actual age in terms of body composition comprehensive index.

### Example 12

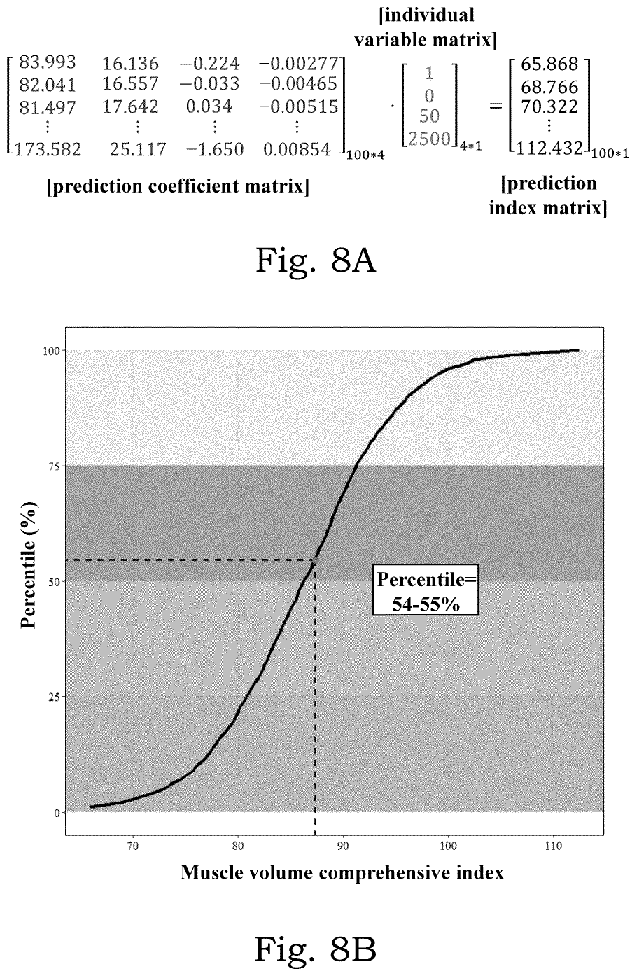

The prediction module (2) produced an individual variable matrix (M2) as shown in FIG. 8A according to individual information provided in Example 7. Parameter values from the 1^{st} to the 4^{th} dimensions in the individual variable matrix (M2) were 1 (prediction variable), 0 (female), 50 (age) and 2500 (Age²). The prediction index matrix (M) was derived from a multiplication of the individual variable matrix (M2) and the prediction coefficient matrix (M1). As shown in FIG. 8B, a curve was plotted by unfolding the prediction index matrix (M), and the predicted percentile of the muscle volume comprehensive index 87.298331 fell among 54 to 55% by machine-based comparison.

According to Examples 6 to 12, the system (100) provided in the present invention enables comprehensive evaluation of the correlation between body composition status and actual age in terms of body composition percentile and body age evaluated value. With reference to the female case, her actual age was 50 years old, and the system (100) judged that her muscle volume comprehensive index fell among 54 to 55% in her age group. This indicates that, on the whole, the comprehensive muscle performance of this female was around mid-level. However, the body age evaluated values derived from 5 distinct comprehensive indices indicated that this female's body age appears to out-perform relative to her actual age, simply from perspectives of muscle volume or muscle quality. In contrast, if internal fat was included in the overall evaluation, the body composition comprehensive index showed that this female underperformed relative to her actual age in terms of body composition. Thus, in view of the foregoing indices, this female could develop a plan of internal fat improvement to narrow the gap between body age and actual age, thereby improving her health by and large.

The method and system provided in the present invention facilitates rapid quantitative analysis and tracking of body composition based on a single medical image. Establishment of a normal model allows prediction of percentile of an individual's body composition in a normal distributed group. Given identical gender, age, body weight or other physiological conditions, the predicted percentile value and body age evaluated value assist in evaluating an individual to be healthy or sub-healthy, assessing the risk of sarcopenia-related diseases, and determining the need for subsequent nutritional or medical intervention.

1. A method for evaluating body composition, comprising:
inputting a body composition index and an individual variable to a body composition prediction model so as to produce a body composition evaluation index, wherein the body composition evaluation index comprises a body composition quantile value or a body age evaluated value, and wherein the body composition index is measured based on a medical image, and comprises a muscle index, a fat index or a comprehensive index, and the individual variable corresponds to an individual variable of the medical image; and
outputting the body composition evaluation index.

2. The method according to claim 1, wherein the body composition prediction model is defaulted to contain m prediction quantile values and a prediction index matrix, wherein the prediction index matrix is a matrix of dimension m*1 comprising m prediction body composition index corresponding to the prediction quantile values one on one, and m is any one of positive integers larger or equal to 1, wherein the method further comprises:
comparing the body composition index and the prediction body composition index based on the prediction index matrix so as to obtain a corresponding predicted quantile value outputted to be the body composition quantile value, wherein:
the prediction index matrix is derived from a multiplication of a prediction coefficient matrix and an individual variable matrix, and the prediction coefficient matrix is a matrix of dimension m*(n+1) comprising a prediction constant corresponding to each one of the prediction quantile values and a prediction coefficient set corresponding to each one of the prediction quantile values, and wherein the prediction coefficient set comprises n prediction coefficients, and n is any one of positive integers larger or equal to 1;
the individual variable matrix is a matrix of dimension (n+1)*1 comprising a prediction variable corresponding to the prediction constant and n sub-variables corresponding to the individual variable.

3. The method according to claim 2, wherein the prediction body composition index is calculated by the following function (I):
$${Q}_{q} ( Y \vert X ) = {β}_{0} ( q ) + {β}_{1} ( q ) {X}_{1} + {β}_{2} ( q ) {X}_{2} + ⋯ + {β}_{n} ( q ) {X}_{n} \qquad ( I )$$
wherein: Q_{g}(Y|X) is the prediction body composition index; X is the individual variable;
Y is a predicted value on condition of X; q is any one of positive integers lower than m;
β₀(q) is the prediction constant; the n prediction coefficients contain β₁(q) to βₙ(q); the n sub-variables contain X₁ to Xₙ.

4. The method according to claim 2, wherein a method for establishing the prediction coefficient matrix comprises:
retrieving a reference image set comprising a plurality of reference medical images;
measuring a reference body composition index corresponding to each one of the reference medical images, and the reference body composition index comprises a reference muscle index or a reference fat index;
extracting a reference individual variable corresponding to each one of the reference medical images, and the reference individual variable comprises n reference sub-variables;
estimating m prediction coefficient sets by conducting a quantile regression analysis based on the reference body composition index and the reference individual variable, wherein any one of the prediction coefficient sets contains a prediction constant and a sub-prediction coefficient set corresponding thereto, and wherein the sub-prediction coefficient set contains n of the prediction coefficients and each one of the prediction coefficients corresponds one-to-one with each one of the reference sub-variables; and
establishing the prediction coefficient matrix based on the prediction coefficient sets and the prediction quantile values.

5. The method according to claim 2, wherein, before the body composition index and the individual variable are input, the method further comprises:
retrieving a median prediction constant and a median prediction coefficient set from the prediction coefficient matrix, and building a body age evaluation function, wherein the median prediction constant corresponds to the prediction constant of a median prediction quantile, and the median prediction coefficient set corresponds to the prediction coefficient set of a median prediction quantile, wherein the body age evaluation function follows a function (II):
$${Q}_{age} = {β}_{0} ( {q}_{me} ) + {β}_{1} ( {q}_{me} ) {X}_{me 1} + {β}_{2} ( {q}_{me} ) {X}_{me 2} + ⋯ + {β}_{n} ( {q}_{me} ) {X}_{men} \qquad ( II )$$
wherein, Q_{age} represents the body composition index, q represents one of any positive integers smaller than m, β₀(qₘₑ) represents the prediction constant, the median prediction coefficient set comprises β₁(qₘₑ) to βₙ(qₘₑ), the Xₘₑ₁ represents a gender variable, and the Xₘₑ₂ to Xₘₑₙ represents body age variables; and
inputting the body composition index and the individual variable to the body age evaluation function to compute the body age evaluated value corresponding thereto, wherein the individual variable comprises the gender variable, and the body age evaluated value is obtained by calculation with the body age variables according to the function (II).

6. The method according to claim 1, wherein:
the muscle index comprises a muscle mass index, a muscle labelling index, and a muscle area index;
the muscle mass index comprises SMI (Skeletal Muscle Index), SMD (Skeletal Muscle Density), ImatA (Intramuscular Adipose Tissue Area), ImatD (Intramuscular Adipose Tissue Density), NamaA (Normal Attenuation Muscle Area), LamaA (Low Attenuation Muscle Area) or a combination thereof;
the muscle labelling index comprises LWM (Lean Whole-body Mass), Total (Total Muscle Mass) or an assigned muscle group selected from a group consisting of lumbar vertebrae side muscle group, leg muscle group, chest muscle group, dorsal muscle group, ventral muscle group, biceps muscle group, triceps muscle group, and core muscle group;
the muscle area index comprises VBA (Vertebral Body Area);
the fat index comprises VatA (Visceral Adipose Tissue Area), VatD (Visceral Adipose Tissue Density), SatA (Subcutaneous Adipose Tissue Area), SatD (Subcutaneous Adipose Tissue Density) or any combination thereof;
the comprehensive index comprises a muscle volume comprehensive index, a muscle quality comprehensive index, a muscle comprehensive index, a visceral fat index or a body comprehensive index; the n sub-variables of the individual variable is extracted from an individual information corresponding to the medical image, and comprises a gender variable, an age variable or a combination thereof.

7. A system for evaluating body composition, comprising:
an imaging analysis module, configured to measure and output a body composition index based on a medical image, and to extract an individual variable corresponding to the medical image, wherein the body composition index comprises a muscle index, a fat index or a comprehensive index; and
a prediction module, being signal-connected to the imaging analysis module, configured of a body composition prediction model, wherein the body composition index and the individual variable are input to the body composition prediction model so as to produce a body composition evaluation index, wherein:
the body composition evaluation index comprises a body composition quantile value or a body age evaluated value;
the muscle index comprises a muscle mass index, a muscle labelling index, and a muscle area index; the muscle mass index comprises SMI (Skeletal Muscle Index), SMD (Skeletal Muscle Density), ImatA (Intramuscular Adipose Tissue Area), ImatD (Intramuscular Adipose Tissue Density), NamaA (Normal Attenuation Muscle Area), LamaA (Low Attenuation Muscle Area) or a combination thereof; the muscle labelling index comprises LWM (Lean Whole-body Mass), Total (Total Muscle Mass) or an assigned muscle group selected from a group consisting of lumbar vertebrae side muscle group, leg muscle group, chest muscle group, dorsal muscle group, ventral muscle group, biceps muscle group, triceps muscle group, and core muscle group; the muscle area index comprises VBA (Vertebral Body Area);
the fat index comprises VatA (Visceral Adipose Tissue Area), VatD (Visceral Adipose Tissue Density), SatA (Subcutaneous Adipose Tissue Area), SatD (Subcutaneous Adipose Tissue Density) or any combination thereof;
the comprehensive index comprises a muscle volume comprehensive index, a muscle quality comprehensive index, a muscle comprehensive index, a visceral fat index or a body comprehensive index;
the n sub-variables of the individual variable are extracted from an individual information corresponding to the medical image, and comprises a gender variable, an age variable or a combination thereof.

8. The system according to claim 7, wherein the body composition prediction model is defaulted to contain m prediction quantile values and m is any one of positive integers larger or equal to 1, and the body composition prediction model comprises:
a prediction coefficient matrix, being a matrix of dimension m*(n+1), comprising a prediction constant corresponding to each one of the prediction quantile values and a prediction coefficient set corresponding to each one of the prediction quantile values, wherein the prediction coefficient set comprises n prediction coefficients, and n is any one of positive integers larger or equal to 1;
an individual variable matrix, being a matrix of dimension (n+1)*1, comprising a prediction variable corresponding to the prediction constant and n sub-variables corresponding to the individual variable; and
a prediction index matrix, being a multiplication product of the prediction coefficient matrix and the individual variable matrix, wherein the prediction index matrix is a matrix of dimension m*1 comprising a prediction body composition index corresponding one-to-one with the prediction quantile numbers, wherein the prediction module further compares the body composition index and the prediction body composition index so as to obtain a corresponding predicted quantile value, and outputs the corresponding predicted quantile value to be the body composition quantile value.

9. The system according to claim 8, further comprising a training module, being connected to the imaging analysis module and the prediction module, configured to establish the prediction coefficient matrix, wherein:
the imaging analysis module obtains a reference body composition index and a reference individual variable based on any one of reference medical images of a reference image set, wherein the reference body composition index comprises a reference muscle index or a reference fat index, and the reference individual variable comprises n reference sub-variables;
the training module conducts a quantile regression analysis based on the reference body composition index and the reference individual variable so as to estimate m prediction coefficient sets, wherein any one of the prediction coefficient sets comprises a prediction constant and a sub-prediction coefficient set, and wherein the sub-prediction coefficient set comprises n of the prediction coefficients, and each of the prediction coefficients corresponds one-to-one with each of the reference sub-variables; and
the training module establishes the prediction coefficient matrix based on the prediction coefficient sets and the prediction quantile values.

10. The system according to claim 9, wherein:
the prediction body composition index is calculated by the following function (I):
$${Q}_{q} ( Y \vert X ) = {β}_{0} ( q ) + {β}_{1} ( q ) {X}_{1} + {β}_{2} ( q ) {X}_{2} + ⋯ + {β}_{n} ( q ) {X}_{n} \qquad ( I )$$
wherein: Q_{q} (Y|X) is the prediction body composition index; X is the individual variable;
Y is a predicted value on condition of X; q is any one of positive integers lower than m;
β₀(q) is the prediction constant; the n prediction coefficients contain β₁(q) to βₙ(q); the n sub-variables contain X₁ to Xₙ; or
the prediction module is further configured of a body age evaluation unit, and body age evaluation unit retrieves a median prediction constant and a median prediction coefficient set from the prediction coefficient matrix, and builds a body age evaluation function, wherein the median prediction constant corresponds to the prediction constant of a median prediction quantile, and the median prediction coefficient set corresponds to the prediction coefficient set of a median prediction quantile, wherein the body age evaluation function follows a function (II):
$${Q}_{age} = {β}_{0} ( {q}_{me} ) + {β}_{1} ( {q}_{me} ) {X}_{me 1} + {β}_{2} ( {q}_{me} ) {X}_{me 2} + ⋯ + {β}_{n} ( {q}_{me} ) {X}_{men} \qquad ( II )$$
wherein, Q_{age} represents the body composition index, q represents one of any positive integers smaller than m, β₀(qₘₑ) represents the prediction constant, and the median prediction coefficient set comprises β₁(qₘₑ) to βₙ(qₘₑ), the individual variable comprises a gender variable Xₘₑ₁ and sub-variables Xₘₑ₄ to Xₘₑₙ, and the Xₘₑ₂ to Xₘₑ₃
